(19) United States
(12) Patent Application Publication
(10) Pub. No.: US 2011/0129436 A1
(43) Pub. Date: Jun. 2, 2011

(54) POLYGLYCEROL SEBECATE PERITONEAL ADHESION PREVENTION BARRIER

(75) Inventors: Howard I. Pryor, II, Brookline, MA (US); Cathryn A. Sundback, Harvard, MA (US); Joseph P. Vacanti, Winchester, MA (US)

(73) Assignee: THE GENERAL HOSPITAL CORPORATION, Boston, MA (US)

(21) Appl. No.: 12/867,528

(22) PCT Filed: Feb. 17, 2009
(86) PCT No.: PCT/US09/34276

§ 371 Date: Feb. 15, 2011

Related U.S. Application Data
(60) Provisional application No. 61/130,076, filed on May 28, 2008.
(60) Provisional application No. 61/029,050, filed on Feb. 15, 2008.

(51) Int. Cl. A61K 31/765; A61P 43/00

(52) U.S. Cl. 424/78.37

(57) ABSTRACT

The present invention is directed to a method of preventing adhesions between two tissue surfaces. The method includes providing a film comprising a condensation polymer of glycerol and a diacid, wherein the film does not contain anti-inflammatory drugs and positioning the film between a first tissue surface and a second tissue surface under conditions effective to prevent adhesion between said first tissue surface and said second tissue surface.

### CROSS REFERENCE TO RELATED APPLICATIONS

This application claims the benefit under 35 U.S.C. §119(e) of U.S. provisional application No. 61/020,190 filed Jan. 10, 2008, and U.S. provisional application No. 61/130, 076 filed May 28, 2008, the contents of which are herein incorporated by reference in their entirety.

### GOVERNMENT SUPPORT

The subject matter of this application was made with support from the United States Government under _____, Grant No. ______. The U.S. Government has certain rights.

### FIELD OF THE INVENTION

This invention relates to methods of using polymer films for preventing adhesions between two tissue surfaces.

### BACKGROUND OF THE INVENTION

Adhesion formation, the joining of two normally separate surfaces due to trauma or inflammation, is a major problem following surgical procedures. Adhesions following surgery frequently cause postoperative pain, blockage of intestines, and infertility. Adhesions are the major cause of intestinal obstruction. Intestinal obstruction caused by adhesions leads to prolonged hospital stays, additional abdominal surgery, and even death. Abnormal scarring in the abdomen also increases the morbidity of future surgery because adhesions lead to increased blood loss and injury to internal organs. Adhesion formation is also problematic in orthopedic and plastic surgeries, such as in the hand, where impediment of movement is frequently troublesome to the patient.

Intra-abdominal adhesions are the leading cause of secondary infertility and responsible for up to 20% of infertility cases. See Ray, N. F., et al., J. Am. Coll. Surg. 186: 1 (1998) and Ellis, H., et al., Lancet 353: 1476 (1999). Abnormal scarring in the abdomen increases the morbidity of future surgery because adhesiolysis may lead to increased blood loss and injury to internal organs. Adhesion formation may also cause additional morbidity in extra-abdominal procedures, such as in the hand, where impediment of movement is frequently troublesome to the patient. The prevention of adhesions would profoundly decrease morbidity and reduce health care costs across a broad range of medical disciplines. See Menzies, D., et al., Ann R Coll Surg Engl., 83:40-6 (2001).

After injury to the peritoneal lining, the entire epithelial lining becomes re-epithelialized with mesothelial cells and is complete in 5-6 days. Peritoneal injury may result in local ischemia, deposition of polymorphonuclear leukocytes, macrophages, fibrin, mesenchymal cells, fibroblasts, and new blood vessels resulting in adhesion formation. Eventually the adhesion matures into a mesothelial covered fibrous band. See DiZerega, G. S. and Campeau, J. D., Hum. Reprod Update 7: 547, 2001.

Open abdominal surgery results in the formation of peritoneal adhesions in nearly 95% of cases. The formation of viscero-parietal (VP) peritoneal adhesions leads to serious post-operative complications. One proposed method to prevent the formation of adhesions is the interposition of a barrier between damaged areas until adhesion-free healing has occurred. See diZerega G. S., Eur J Surg Suppl 1997,577:10-16 (1997). Several products currently exist that attempt to address the issue of VP adhesions including Seprafilm, Interceed and a variety of injectable gels, however all clinically available barrier products are fraught with drawbacks including poor degradability, variable efficacy and difficulty of use. Seprafilm, the most commonly used clinically available product, is notorious for the difficulty of its application and once it makes contact with tissue it cannot be repositioned. Such a bather must be non-inflammatory, non-immunogenic, biodegradable, easy to use, and must remain as a barrier for at least 14 days. See Risberg, B., Eur J Surg Suppl 1997, (577):32-39 (1997).

The present invention is directed to overcoming these and other deficiencies in the art.

### BRIEF DESCRIPTION OF THE DRAWINGS

FIG. 1 schematically illustrates various embodiments of an elastomeric composition of the present inventions.

FIG. 2 schematically illustrates various embodiments of an elastomeric composition of the present inventions.

FIGS. 3A-D schematically illustrates a formation scheme for an elastomeric composition or material according to various embodiments of the present inventions. FIG. 3A illustrating polycondensation of glycerol and sebacic acid, to form a pre-polymer (a low molecular weight polymer is illustrated), where R is H, and alkyl, alkenyl, or alkynyl). FIG. 3B illustrating functionalization of the pre-polymer backbone with a vinyl group, here acrylation is shown. FIGS. 3C and 3D schematically illustrate examples of portions of the polymer network formed in a various embodiments of a cross-linked polymer of PGSA.

FIG. 4 schematically illustrates an example of the portion of the polymer network formed in a various embodiments of a cross-linked polymer of PGSA-PEG.

FIG. 5 is a graph of the comparison of adhesion formation of sham and PGS film after cecal and abdominal wall abrasion experiments.

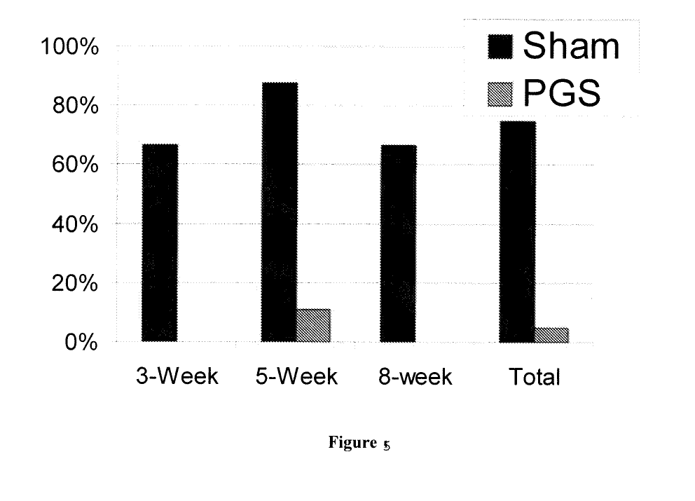

FIG. 6 is a graph of adhesion severity. See Kutlay J, et al. “Comparative Effectiveness of Several Agents for Preventing Postoperative Adhesions.” World J Surg (2004) 28, 662-665, which is hereby incorporated by reference in its entirety.

### SUMMARY OF THE INVENTION

One aspect of the present invention is directed to a method of preventing adhesions between two tissue surfaces. The method includes providing a film comprising a condensation polymer of glycerol and a diacid, wherein the film does not contain anti-inflammatory drugs and positioning the film between a first tissue surface and a second tissue surface under conditions effective to prevent adhesion between said first tissue surface and said second tissue surface.

Another aspect of the present invention is directed to a method of preventing adhesions between two tissue surfaces. The method includes providing a film consisting essentially of a condensation polymer of glycerol and a diacid and positioning the film between a first tissue surface and a second tissue surface under conditions effective to prevent adhesion between said first tissue surface and said second tissue surface.

Open abdominal surgery results in the formation of peritoneal adhesions in nearly 95% of cases. The formation of viscero-parietal (VP) peritoneal adhesions is a direct cause of small bowel obstruction (SBO); significantly increases the risk of enterotomy, bleeding and bladder injury in any future surgical intervention; and can eliminate the option of laparoscopy in many cases. One method to prevent the formation of adhesions is the interposition of a barrier between damaged areas until adhesion-free healing has occurred. Such a barrier must be non-inflammatory, non-immunogenic, biodegradable, easy to use, and must remain as a barrier for at least 14 days. A biodegradable polymer film, as described in International Patent Publication No. WO 03/064496, meets all of these criteria and was evaluated in the rat model as a barrier to prevent the formation of VP adhesions. Such a barrier would be especially beneficial for patients who are prone to adhesions and re-operation, such as those with Crohn's disease or acute diverticulitis requiring diversion and an eventual reversal of a colostomy. Also provided are methods of using the compositions described herein to prevent adhesions between tissues. Also described herein are uses of the compositions described herein in the manufacture of a medicament to prevent adhesions between tissue surfaces.

### DETAILED DESCRIPTION OF THE INVENTION

Adhesions develop at sites of peritoneal trauma caused by typical surgical maneuvers such as incision, retraction, and cauterization, as well as, from desiccation and ischemia. Such peritoneal trauma results in a defect in the mesothelial layer of the peritoneum exposing the underlying tissue Initially, the defect is coated by fibrin which causes the surface to become “sticky.” Normal healing depends on effective clearance of the fibrin coating by an enzyme called plasmin. If plasmin adequately clears the fibrin coating, a mesothelial defect will close within 7 days. However, when the fibrin coating is not removed adequately the surface remains “sticky” and when two such surfaces come into contact they adhere to one another. Once these sites of adherence develop, the remaining fibrin coat is reorganized into connective tissue and a mature post-operative adhesion is formed. Adhesions that form between the visceral peritoneum and the parietal peritoneum (V-P adhesions) account for up to 84% of post-operative adhesions and may pose special risks.

V-P adhesions may be a principal cause of chronic post-operative abdominal pain. The parietal peritoneum is unique because it contains nerve endings that respond to thermal, chemical and mechanical stimulation whereas, the visceral peritoneum only responds to tension stimuli. When patients were asked to rate their pain from adhesions formed between the peritoneum of a mobile organ (ovary, bladder, small bowel) and the parietal peritoneum they consistently reported higher scores when compared to the pain from adhesions between two separate portions of the bowel. Despite the persistent nature of this type of pain it is typically recommended that no operative invention be undertaken unless clinically mandated by a concern of compromised bowel because simple lysis of adhesions will merely result in the re-formation of adhesions which may involve additional intra-abdominal structures.

Adhesions involving the small bowel are considered a primary cause of small bowel obstruction (SBO) and V-P adhesions between the small bowel and the abdominal wall have been demonstrated in up to 63% of patients following a single operation. The percentage of patients with these type of adhesions that will develop SBO remains unknown, however, one-third of all patients with an SBO will require an operation to divide adhesions. The mortality associated with SBO operations is 3% and an additional 8% will face significant post-operative complications. Of those requiring surgery, approximately 30% will experience a recurrence of symptoms and up to 7% of those patients will require another operation.

The omentum forms adhesions to the abdominal wall far more frequently than the small bowel (72% vs. 18%) and together adhesions involving these organs complicate subsequent operations and may limit the future use of laparoscopic techniques. The lysis of V-P adhesions at re-operation has been shown to extend the operative time up to 57 minutes. Re-operations in the presence of V-P adhesions can be further complicated by inadvertent enterotomy (IE). Repeat laparotomy is associated with a 20% IE rate that increases to a rate of 33% when the re-operation is for recurrent SBO. Between 10 and 18% of cases involving V-P adhesions require conversion to laparotomy which is associated with increased perioperative complications such as enterocutaneous fistula, and longer hospital stays. The key to avoiding perioperative adhesion complications including chronic pain, SBO, difficult re-operation and in advertent enterotomy is to prevent the formation of V-P adhesions.

A host of strategies have been proposed to prevent adhesion formation including meticulous surgical technique, topical application fibrinolytic agents, crystalloid irrigation solutions, and systemic anti-inflammatory agents, however, the typical clinical approach is to position a barrier between two injured areas of peritoneum to prevent apposition. The ideal characteristics of an anti adhesion bather include: anti-adhesive efficacy; biocompatibility; resorbability; efficacy on an oozing surface; and laparoscopic applicability. Several products have been evaluated as anti-adhesion barriers and the most successful of these is Seprafilm (Genzyme, Cambridge, Mass.). Serprafilm has been demonstrated to show anti-adhesive efficacy; biocompatibility; resorbability; and efficacy on an oozing surface. However, Seprafilm it is difficult to handle in the operating room because it sticks to moist surfaces. In addition, Seprafilm cannot be repositioned once applied and will stick inadvertently to an abdominal retractor or surgical gloves. Up to 20% of the material in one study was rendered useless due to handling difficulties before being applied to the target organ. Seprafilm is even more challenging to use laparoscopically requiring special equipment; advanced laparoscopic skills, and the insertion and retrieval of covering materials. Given the vast health care implications of V-P adhesions, the limited bather prevention options, and the absence of a definitive laparoscopic treatment for adhesion related disease a need remains for an effective method to prevent V-P adhesions.

In general, PGS or other films described herein (“films”) can be used as a barrier to separate two inflamed tissues as they heal. This separation prevents the formation of adhesions and maintains the anatomic planes between the two tissues. Specific examples include:
1) Thoracic cavity
   a. Lung Resection—Films could be used to prevent lung-to-pleural scarring following any normal resection of lung either by video assisted thoracosopic surgery (VATS) or open thoracotomy.
   b. Decortication—The treatment of empyema involves an operation to remove the inflammatory rind from the effected lung. The use of a film could prevent the formation of adhesions between the lung and the pleural lining following decortication.
   c. Films could be used to coat mesh for repair of congenital diaphragmatic hernia. This would allow the mesh to develop a mesothelial lining during the healing process and could widen the number of mesh products available for this challenging repair.
2) Mediastinum
   a. Cardiac—Films could be used to prevent adhesion formation between the cardiac muscle and surrounding mediastinal structures following open or minimally invasive cardiac surgery.
   b. Esophagus
      i. TEF—Films could be used to keep the tissue planes separated following the repair of a tracheal-esophageal fistula.
      ii. Espohageal Perforation—Films can be used to keep the tissue planes separated following esophageal perforation repair.
3) Abdomen
   a. Simple Adhesion Prevention—Any patient undergoing open or laparoscopic abdominal surgery could potentially benefit from the use of a film to prevent the formation of post-operative adhesions
   b. Trauma—Damage control surgeries require frequent re-operations during the same hospital admission. Typically, patients are sent to the ICU without formal closure of their abdomen. Films could be used in this setting as a bather to evisceration that generates minimal inflammatory response until formal closure can be performed.
   c. Entero-Cutaneous Fistula—EC-fistulas complicate many operations that involve local inflammation. Films could be used as a preventative measure in cases where the risk of EC fistula formation is especially high. In addition, once formed the treatment for EC fistulas is to separate the tissue planes and close the fistula. These procedures fail frequently and could benefit from films as a separating device.
   d. Anticipated re-operation—Any procedure that involves a second procedure weeks or months later (i.e. Hartmann's procedure) would benefit from the use of a film to minimize adhesion formation and simplify the second procedure's abdominal access.
   e. Ventral Hernia Repair—Films could be used to coat mesh for repair of a ventral hernia. This would allow the mesh to develop a mesothelial lining during the healing process and could widen the number of mesh products available for this challenging repair.
   f. AAA Graft Protection—Films could be used to protect the graft and closure of an open abdominal aortic aneurysm repair. These repairs are occasionally complicated by the formation of an entero-aortic fistula. This complication is frequently lethal as the patient exsanguinates via the bowel and for those that survive often the graft is seeded with bowel. Preventing this complication with a film may be useful.
4) Pelvis
   a. Endometriosis—Endometriosis is a disease characterized by the retrograde seeding of the peritoneal cavity with the deciduous endometrial lining of the uterus. These seedings cause significant localized inflammation and scarring. Since these seedings grow and recede in response to the menstrual cycle they cause repeated pain. If medical treatment fails, the treatment is thermal ablation by laparoscopy which results in another round of scarring. Following an ablation, films could be used to prevent adhesion formation between the ablated tissue and associated peritoneum.
   b. Pelvic Inflammatory Disease—Pelvic inflammatory disease results in scarring and distortion of the female reproductive tract which can increase the risk of ectopic pregnancy and result in infertility. Diagnostic laparoscopy is frequently used to release the scars and allow the anatomy to return to its normal position, however, the procedure has a high failure rate due to recurrence of scarring. Films could prevent the two apposed tissue planes from coming into contact during healing.
5) Central Nervous System—Films could be used to prevent the formation of adhesions between the dura and any epidural structure following neurosurgical procedures.

Poly(Glycerol Sebacate) (PGS) was first introduced by Langer et al. in 2002 as a tough and inexpensive biodegradeable elastomer with excellent biocompatibility. It is inexpensive, non-immunogenic, and endotoxin-free. It is composed of glycerol and sebacic acid, two materials approved by the FDA for use in medical devices and has been shown to retain its mechanical strength and geometry better than other biodegradable materials. Preliminary in vivo studies have demonstrated that PGS induces a minimal inflammatory response and virtually no capsule formation in subcutaneous implantation studies.

Biodegradable films include elastomeric polymers, copolymers, and films including poly(glycerol sebacate) (PGS); poly(glycerol sebacate apidicate) PGSA; and poly(glycerol sebacate)-acrylate-co-poly(ethylene glycol) (PGSA-PEG), and networks and derivatives thereof. Examples and synthesis procedures are found in International Patent Application No.: PCT/US2007/060529 and United States Patent Application No.: 20090011486, and references therein, which are hereby incorporated by reference in their entirety.

One aspect of the present invention is directed to a method of preventing adhesions between two tissue surfaces. The method includes providing a film comprising a condensation polymer of glycerol and a diacid, wherein the film does not contain anti-inflammatory drugs and positioning the film between a first tissue surface and a second tissue surface under conditions effective to prevent adhesion between said first tissue surface and said second tissue surface.

In a preferred embodiment, the diacid is sebacic acid and the polymer is polyglycerol sebacate.

In certain embodiments, the first tissue surface, and/or second tissue surface is a site of surgical activity. In other embodiments, the first tissue and/or second tissue is visceral tissue. In a preferred embodiment, the first tissue and/or second tissue is peritoneal tissue. In a more preferred embodiment, the adhesions prevented are viscero-parietal peritoneal adhesions.

Another aspect of the present invention is directed to a method of preventing adhesions between two tissue surfaces. The method includes providing a film consisting essentially of a condensation polymer of glycerol and a diacid and positioning the film between a first tissue surface and a second tissue surface under conditions effective to prevent adhesion between said first tissue surface and said second tissue surface.

In a preferred embodiment, the diacid is sebacic acid and the polymer is polyglycerol sebacate.

In certain embodiments, the first tissue surface, and/or second tissue surface is a site of surgical activity. In other embodiments, the first tissue and/or second tissue is visceral tissue. In a preferred embodiment, the first tissue and/or second tissue is peritoneal tissue. In a more preferred embodiment, the adhesions prevented are viscero-parietal peritoneal adhesions.

A barrier film, for example PGS, can be used to prevent viscero-parietal peritoneal abdominal adhesions for both laparoscopic and open abdominal procedures. A barrier film would be deployed in either instance following the completion of the essential aspects of the procedure and preceding closure. For use in a laparoscopic procedure, the barrier film can be rolled into a thin tube so that it can be passed through a standard laparoscopic trocar and introduced into the abdomen. Once in the abdomen, the film can be unrolled and positioned to prevent contact between the visceral and parietal peritoneal surfaces. The ability to reposition this film makes it expressly useful in laparoscopic procedures. The application for open abdominal procedures would be essentially the same except that the film can be applied as a sheet without having to be rolled.

Efficacy of treatment is determined by the absence of development of post-operative adhesions.

PGS or other film may be modified to incorporate bioactive agents, e.g., fibrinolytics or other agents, either directly or encapsulated into microspheres. The microspheres may be released in a controlled manner, for example, over the first 2-3 days. Fibrinolytics like tissue plasminogen activator (tPA) enhance lysis of fibrin, a critical component in adhesion formation. In addition, the nonadhesive wetted surface character of certain films, including PGS, which allows facile positioning, may permit PGS films to move under gravitational force when implanted in the peritoneal cavity of certain subjects.

PGS may be polymerized photochemically instead of thermally (see Nijst C L, et al. Biomacromolecules 2007 October; 8(10):3067-3073, which is hereby incorporated by reference in its entirety). Photochemical curing allows bioactive molecules, either alone or encapsulated in microspheres, to be incorporated into the PGS polymer, while maintaining their biological activity. This polymerization platform will also support surface modifications to increase adhesiveness.

Incorporation of the gecko-inspired nanotopography with a surface layer of oxidized dextran glue, which has been shown to be effective in bonding PGS to tissue (see Burns, et al., Eur J Surg Suppl 1997(577):40-48, which is hereby incorporated by reference in its entirety); the rate of PGS-tissue bonding is slow enough to allow facile placement and repositioning of PGS films.

Another aspect of the present invention is directed to a method of preventing adhesions between two tissue surfaces. The method includes providing a film substantially the same as any of the films described herein, wherein the film does not contain anti-inflammatory drugs and positioning the film between a first tissue surface and a second tissue surface under conditions effective to prevent adhesion between said first tissue surface and said second tissue surface.

In certain embodiments, the film may include surface-deposits of a material designed to enhance the bonding of the film to a tissue. For example, oxidized dextran glue. The material or glue may be applied as a layer or in a pattern or in discreet locations, as desired.

In certain embodiments, the film may include bioactive agents, drugs, or other molecules, either alone or encapsulated in microspheres, to be incorporated into the film. For example, fibrinolytics like tissue plasminogen activator (tPA) may be used.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein, the article “a” is used in its indefinite sense to mean “one or more” or “at least one.” That is, reference to any element of the present teachings by the indefinite article “a” does not exclude the possibility that more than one of the element is present.

The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

The term “biocompatible”, as used herein is intended to describe materials that do not elicit a substantial detrimental response in vivo.

As used herein, “biodegradable” polymers are polymers that degrade down to monomeric species under physiological or endosomal conditions. In various preferred embodiments, the polymers and polymer biodegradation byproducts are biocompatible. Biodegradable polymers are not necessarily hydrolytically degradable and may require enzymatic action to fully degrade.

As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of examples of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, the entire contents of which are herein incorporated by reference.

As used herein, the term “tissue” refers to a collection of similar cells combined to perform a specific function, and any extracellular matrix surrounding the cells.

The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/.about.dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionaliwition, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

The term “substituted” is intended to describe groups having substituents replacing a hydrogen on one or more atoms, e.g., carbon, nitrogen, oxygen, etc., of a molecule. Substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkoxyl, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, nitro, trifluoromethyl, cyano, azido, heterocyclyl, alkylaryl, or an aromatic or heteroaromatic group. Accordingly, the phrase “a substituent as described herein” or the like refers to one or more of the above substituents, and combinations thereof.

The term “alkyl” includes saturated aliphatic groups, which includes both “unsubstituted alkyls” and “substituted alkyls”, the latter of which refers to alkyl groups having substituents replacing a hydrogen on one or more carbons of the hydrocarbon backbone. The term “alkyl” includes straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl(alicyclic) groups (cyclopropyl, cyclopentyl, cyclohexyl, cycloheptyl, cyclooctyl), and cycloalkyl substituted alkyl groups. The term “alkyl” also includes the side chains of natural and unnatural amino acids.

An “alkylaryl” or an “aralkyl” group is an alkyl substituted with an aryl (e.g., phenylmethyl(benzyl)).

The term “aryl” includes 5- and 6-membered single-ring aromatic groups, as well as multicyclic aryl groups, e.g., tricyclic, bicyclic, e.g., naphthalene, anthracene, phenanthrene, etc.). The aromatic ring(s) can be substituted at one or more ring positions with such substituents as described above. Aryl groups can also be fused or bridged with, e.g., alicyclic or heterocyclic rings which are not aromatic so as to form, e.g., a polycycle.

The term “alkenyl” includes unsaturated aliphatic groups analogous in length and possible substitution to the alkyls described above, but which contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl(alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl includes both “unsubstituted alkenyls” and “substituted alkenyls”, the latter of which refers to alkenyl groups having substituents replacing a hydrogen on one or more carbons of the hydrocarbon backbone.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentyl, hexynyl, heptynyl, octynyl, nonynyl, decynyl, etc.), branched-chain alkynyl groups, and cycloalkyl or cycloalkenyl substituted alkynyl groups. The term alkynyl includes both “unsubstituted alkynyls” and “substituted alkynyls”, the latter of which refers to alkynyl groups having substituents replacing a hydrogen on one or more carbons of the hydrocarbon backbone.

The term “acyl” includes compounds and groups which contain the acyl radical (CH.sub.3CO—) or a carbonyl group. The term “substituted acyl” includes acyl groups having substituents replacing a one or more of the hydrogen atoms.

The term “acylamino” includes groups wherein an acyl group is bonded to an amino group. For example, the term includes alkylcarbonylamino, arylcarbonylamino, carbamoyl and ureido groups.

The term “aroyl” includes compounds and groups with an aryl or heteroaromatic group bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group that is bound to at least one alkyl group and at least one aryl group. The term “alkaminoalkyl” refers to an alkyl, alkenyl, or alkynyl group bound to a nitrogen atom that is also bound to an alkyl group.

The term “amide” or “aminocarboxy” includes compounds or groups that contain a nitrogen atom that is bound to the carbon of a carbonyl or a thiocarbonyl group. The term includes “alkaminocarboxy” groups that include alkyl, alkenyl, or alkynyl groups bound to an amino group bound to a carboxy group. It includes arylaminocarboxy groups that include aryl or heteroaryl groups bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include groups wherein alkyl, alkenyl, alkynyl and aryl groups, respectively, are bound to a nitrogen atom which is in turn bound to the carbon of a carbonyl group.

The term “carbonyl” or “carboxy” includes compounds and groups which contain a carbon connected with a double bond to an oxygen atom, and tautomeric forms thereof. Examples of groups that contain a carbonyl include aldehydes, ketones, carboxylic acids, amides, esters, anhydrides, etc. The term “carboxy group” or “carbonyl group” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl group. For example, the term includes groups such as, for example, aminocarbonyl groups, (wherein a nitrogen atom is bound to the carbon of the carbonyl group, e.g., an amide), aminocarbonyloxy groups, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. groups.

The term “ether” includes compounds or groups that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and groups that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “hydroxy” or “hydroxyl” includes groups with an —OH or —O.sup.-.

The term “halogen” includes fluorine, bromine, chlorine, iodine, etc. The term “perhalogenated” generally refers to a group wherein all hydrogens are replaced by halogen atoms.

The term “heteroatom” includes atoms of any element other than carbon or hydrogen. Preferred heteroatoms are nitrogen, and oxygen. The term “heterocycle” or “heterocyclic” includes saturated, unsaturated, aromatic (“heteroaryls” or “heteroaromatic”) and polycyclic rings which contain one or more heteroatoms. The heterocyclic may be substituted or unsubstituted. Examples of heterocyclics include, for example, benzodioxazole, benzofuran, benzoimidazole, benzothiazole, benzothiophene, benzoxazole, chromene, deazapurine, furan, indole, indolizine, imidazole, isoxazole, isoindole, isoquinoline, isothiaozole, methylenedioxyphenyl, napthridine, oxazole, purine, pyran, pyrazine, pyrazole, pyridazine, pyridine, pyrimidine, pyrrole, quinoline, tetrazole, thiazole, thiophene, and triazole. Other heterocycles include morpholino, piprazine, piperidine, thiomorpholino, and thioazolidine.

The terms “polycyclic ring” and “polycyclic ring structure” include groups with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycyclic ring can be substituted with such substituents as described above.

In various aspects, the present inventions provide elastic biodegradable polymer compositions and materials formed by the reaction of a multifunctional alcohol or ether (that is a compound having two or more OR groups, where each R is independently H and an alkyl) and a difunctional or higher order acid (e.g., a diacid) to form a pre-polymer, which is cross-linked to form the elastic biodegradable polymer. In preferred embodiments, the cross-linking is performed by functionalization of one or more OR groups on the pre-polymer backbone with vinyl, followed by photopolymerization to form the elastic biodegradable polymer composition or material. Preferably, acrylate is used to add one or more vinyls to the backbone of the pre-polymer to form an acrylated pre-polymer.

### Biodegradable Adhesion-Prevention Barrier Elastomeric Polymers

For practice of the present invention it is contemplated that the following films can be used.

In various embodiments, the compositions and materials of the present inventions can be formed from a relatively inexpensive biodegradable photocurable elastomer, poly(glycerol sebacate apidicate) PGSA. In various embodiments, the compositions and materials of the present inventions can be formed in seconds via photopolymerization, facilitating, e.g., their formation in situ. In various embodiments, compositions and materials of the present inventions are formed from viscous liquid acrylated pre-polymer, facilitating the molding and/or injection of the acrylated pre-polymer to form materials, structures and various devices. In addition, in various embodiments, the photoinitiated crosslinking reaction used to form the compositions and materials of the present invention, does not require a solvent.

In various aspects, the present inventions provide elastomeric compositions comprising a cross-linked polyester; the cross-linked polyester comprising a polymeric unit of the general formula (-A-B-).sub.n where, n represents an integer greater than 1, A represents a substituted or unsubstituted ester and B represents a substituted or unsubstituted acid ester comprising at least two acid ester functionalities. At least a portion of the cross-links between polymeric units forming a dioic acid ester between the A components.

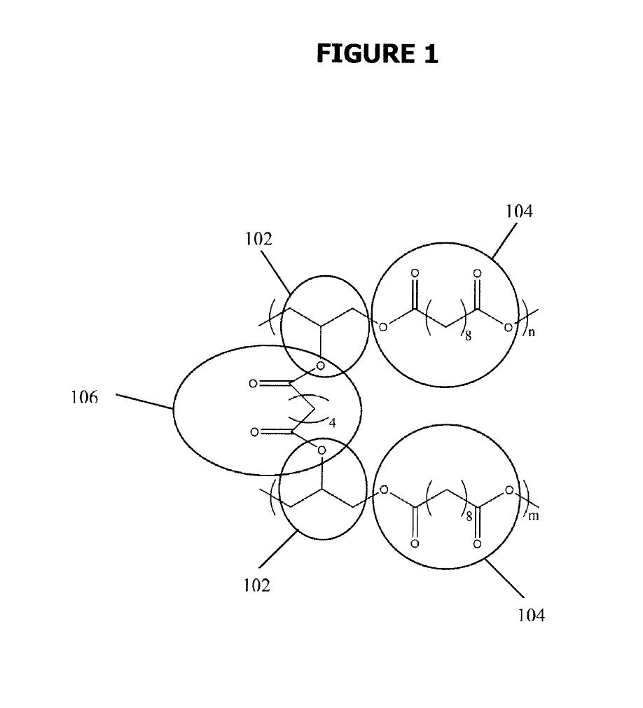

Referring to FIG. 1, various embodiments of an elastomeric composition a which comprises a repeating polymeric unit of the general formula (-A-B-).sub.n are illustrated; the A component including a substituted or unsubstituted ester (102), the B component including a substituted or unsubstituted acid ester comprising at least two acid ester functionalities (104), and the cross-link forming a dioic acid ester (106) between at least a portion of the A components (102).

In various embodiments, these elastomeric compositions comprise a portion that can be represented by the general formula (I) below, where m, n, p, q, and v are each independently integers greater than 1.

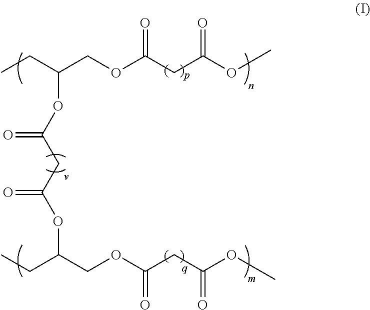

In various preferred embodiments, an elastomeric composition represented by general formula (I) is derived from cross-linking poly(glycerol sebacate)-acrylate (PGSA) using UV excitation in the presence of a photoiniator (or other free radical initiated systems) of the acrylate to initiate the cross-linking reaction. In various embodiments of the methods of the present invention, one or more hydrogel or other polymeric precursors (e.g., precursors that may be modified to contain acrylate groups such as poly(ethylene glycol), dextran, chitosan, hyaluronic acid, alginate, other acrylate based precursors including, for example, acrylic acid, butyl acrylate, 2-ethylhexyl acrylate, methyl acrylate, ethyl acrylate, acrylonitrile, n-butanol, methyl methacrylate, and TMPTA, trimethylol propane trimethacrylate, pentaerythritol trimethacrylate, pentaerythritol tetramethacrylate, ethylene glycol dimethacrylate. dipentaerythritol penta acrylate, Bis-GMA (Bis phenol A glycidal methacrylate) and TEGDMA (tri-ethylene, glycol dimethacrylate), sucrose acrylate, and combinations thereof, can be reacted with the acrylated pre-polymer (e.g., PGSA) prior to or during free radical polymerization to modify the cross-links between the polymer chains.

In various aspects, the present inventions provide elastomeric compositions comprising a cross-linked polyester; the cross-linked polyester comprising a polymeric unit of the general formula (-A-B-).sub.n cross-linked between at least a portion of the A components of the polyester, the cross-link forming a link comprising at least a portion of the general formula -(D).sub.k-C—; where A represents a substituted or unsubstituted ester, B represents a substituted or unsubstituted acid ester comprising at least two acid ester functionalities; C represents a substituted or unsubstituted dioic acid ester; D represents one or more of a substituted or unsubstituted ester, and k is an integer greater than 0 and n an integer greater than 1. It is to be understood that the elastomeric compositions can contain one or more kinds of cross-links in addition to a cross-link comprising a dioic acid ester and an ester.

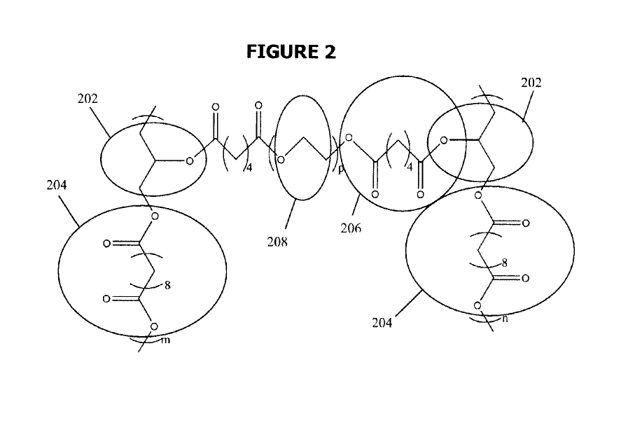

Referring to FIG. 2, various embodiments of an elastomeric composition comprising a repeating polymeric unit of the general formula (-A-B-).sub.n are illustrated; the A component including a substituted or unsubstituted ester (202), the B component including a substituted or unsubstituted acid ester comprising at least two acid ester functionalities (204), and the cross-link forming a substituted or unsubstituted dioic acid ester (206) and a substituted or unsubstituted ester (208) between at least a portion of the A components (202). In various embodiments, the ester linkage forms a polyester, e.g., p in FIG. 2 is an integer greater than 1.

In various embodiments, these elastomeric compositions comprise a portion that can be represented by the general formula (II) below, where k, m, n, p, q, and v are each independently an integer greater than 1.

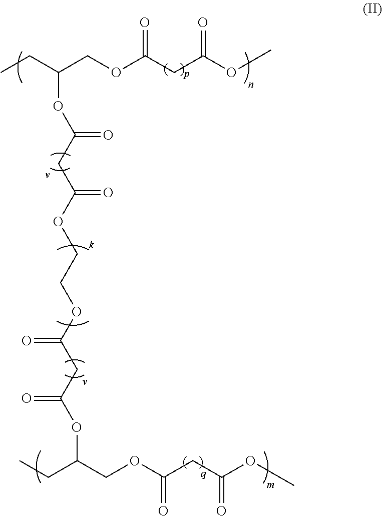

In various preferred embodiments, an elastomeric composition represented by general formula (II) is derived from copolymerization of PGSA with various proportions of an acrylated polyester, e.g., PEGD, to form one or more crosslinks of the general formula -(D).sub.k-C—; where C represents a dioic acid ester, D represents an ester, and k an integer greater than 1, between polymer chains. In various embodiments, by selecting the proportion of PEGD to PGSA the material properties of the elastomeric composition can be selected. For example, in various embodiments, the PGSA-PEG composition can provide a hydrogel material (e.g., equilibrium water content greater than about 30%) with elastic properties.

In various embodiments, the present inventions provide an elastomeric biodegradable material formed from a cross-linked polyester, the elastomeric biodegradable material having a degradation rate that is substantially non-monotonic as a function of overall cross-link density. In various embodiments the degradation rate is the in vitro degradation rate in phosphate buffer saline (PBS), or in acidic or alkaline conditions. In various embodiments the degradation rate is the in vivo degradation rate. In various embodiments, the present inventions provide an elastomeric biodegradable material formed from a cross-linked polyester, the elastomeric biodegradable material having a degradation rate that is capable of being increased by increasing overall cross-link density. In various embodiments, the present inventions provide an elastomeric biodegradable material formed from a cross-linked polyester, the elastomeric biodegradable material having a degradation rate that is capable of being increased without substantially decreasing the tensile Young's modulus of the material.

In various aspects, the present inventions provide methods for forming a biodegradable elastomeric material, comprising the steps of (a) reacting a first component comprising two or more functionalities of the general formula —OR, where R of each group is independently hydrogen or alkyl, with a second component comprising two or more acid ester functionalities to form a mixture of pre-polymers having a molecular weight in the range between about 300 Da and about 75,000 Da; (b) reacting the mixture of pre-polymers with an acrylate to form a mixture of acrylated pre-polymers; and (c) irradiating the acrylated pre-polymer mixture with ultraviolet light to cross-link at least a portion of the acrylated pre-polymers and form a biodegradable elastomeric material; wherein the pre-polymer mixture is not heated above about 45.degree. C. during irradiation, and preferably not above about 37.degree. C., and more preferably not above about 25.degree. C.

In various embodiments, the methods comprise adding one or more additional acrylated molecules (referred to as acrylated co-polymers herein) during the reacting the mixture of pre-polymers with an acrylate, or to the mixture of acrylated pre-polymers. A wide variety of co-polymers can be used including, but not limited to, dextran, hyaluronic acid, chitosan, and poly(ethylene glycol).

In various aspects, the present inventions provide methods for forming a biodegradable elastomeric material, comprising the steps of: (a) providing a solution comprising: a pre-polymer comprising (i) a first component comprising two or more functionalities of the general formula —OR, where R of each group is independently hydrogen or alkyl; and (ii) a second component comprising two or more acid ester functionalities; and (c) crosslinking at least a protion of the pre-polymers using one or more of a Mitsunobu-type reaction, polymerization using a thermal initiator, redox-pair initiated polymerization, and a Michael-type addition reaction using a bifunctional sulfhydryl compound.

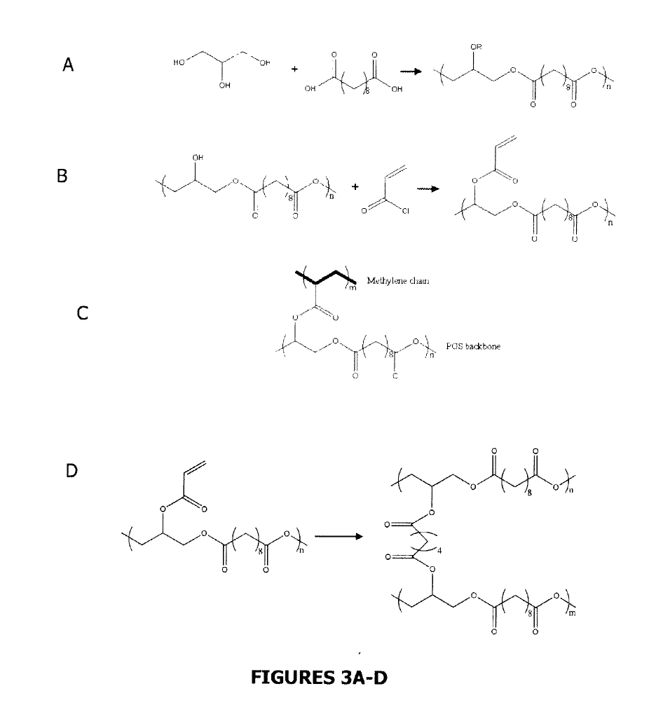

In various aspects, the present inventions provide elastic biodegradable polymer compositions and materials formed by the reaction of a multifunctional alcohol or ether (that is a compound having two or more OR groups, where each R is independently H and an alkyl) and a difunctional or higher order acid (e.g., a diacid) to form a pre-polymer (see, e.g., FIG. 3A), which is cross-linked to form the elastic biodegradable polymer. In preferred embodiments, the cross-linking is performed by functionalization of one or more OR groups on the pre-polymer backbone with vinyl (see, e.g., FIG. 3B), followed by photopolymerization to form the elastic biodegradable polymer composition or material. Preferably, acrylate is used to add one or more vinyls to the backbone of the pre-polymer to form an acrylated pre-polymer.

Referring to FIGS. 3A-D and 4, this formation scheme is schematically illustrated. It is to be understood that the acrylation and polymerization reactions can result in several types of cross-links within the polymer network. For example, the acrylated hydroxyl upon photopolymerization can yield acid ester cross-links to an alkyl chain (also know in the art as a methylene chain) (see, e.g., FIG. 3C), as well as dioic acid ester cross-links when, for example, two acrylated hydroxides react (see, e.g., FIG. 3D.)

### Diacid Component

A wide variety of diacid, or higher order acids, can be used in the formation of a elastic biodegradable polymer compositions and materials according to various embodiments of the present invention, including, but are not limited to, glutaric acid (5 carbons), adipic acid (6 carbons), pimelic acid (7 carbons), suberic acid (8 carbons), and azelaic acid (nine carbons). Exemplary long chain diacids include diacids having more than 10, more than 15, more than 20, and more than 25 carbon atoms. Non-aliphatic diacids can be used For example, versions of the above diacids having one or more double bonds can be employed to produce glycerol-diacid co-polymers. Amines and aromatic groups can be incorporated into the carbon chain. Exemplary aromatic diacids include terephthalic acid and carboxyphenoxypropane. The diacids can also include substituents as well. For example, in various embodiments, reactive groups like amine and hydroxyl can be used increase the number of sites available for cross-linking. In various embodiments, amino acids and other biomolecules can be used to modify the biological properties of the polymer. In various embodiments, aromatic groups, aliphatic groups, and halogen atoms can be used to modify the inter-chain interactions within the polymer.

### Pre-Polymer

In various embodiments, the pre-polymer of the present inventions comprises a diol, or higher order, portion and a diacid, or higher order acid, portion. In various embodiments, the pre-polymer can include unsaturated diols, e.g., tetradeca-2,12-diene4,14-diol, or other diols including macromonomer diols such as, e.g., polyethylene oxide, and N-methyldiethanoamine (MDEA). In addition to incorporating these into the pre-polymer, the diols can be incorporated into the resultant cross-linked polymer through, e.g., acrylate chemistry. For example, the diols could be first acrylated and then combined with acrylated pre-polymer using a free radical polymerization reaction. In various embodiments, aldehydes and thiols can be used, e.g., for attaching proteins and growth factors to the pre-polymer.

### Vinyl Addition to Pre-Polymer

A variety of techniques can be used to functionalize the pre-polymer with vinyl. In various preferred embodiment an acrylate, such as, for example, an acrylate monomer. Examples of suitable acrylate monomers include, but are not limited to, methacrylate, vinyl methacrylate, maleic methacrylate, and those having the structure

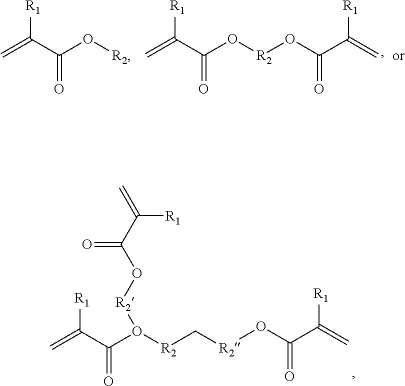

where R.sub.1 can be methyl or hydrogen; and R.sub.2, R.sub.2′, and R.sub.2″ can be alkyl, aryl, heterocycles, cycloalkyl, aromatic heterocycles, multicycloalkyl, hydroxyl, ester, ether, halide, carboxylic acid, amino, alkylamino, dialkylamino, trialkylamino, amido, carbamoyl thioether, thiol, alkoxy, or ureido groups. R.sub.2, R.sub.2′, and R.sub.2″ may also include branches or substituents including alkyl, aryl, heterocycles, cycloalkyl, aromatic heterocycles, multicycloalkyl, hydroxyl, ester, ether, halide, carboxylic acid, amino, alkylamino, dialkylamino, trialkylamino, amido, carbamoyl, thioether, thiol, alkoxy, or ureido groups. Further examples of suitable acrylate monomers include, but are not limited to,

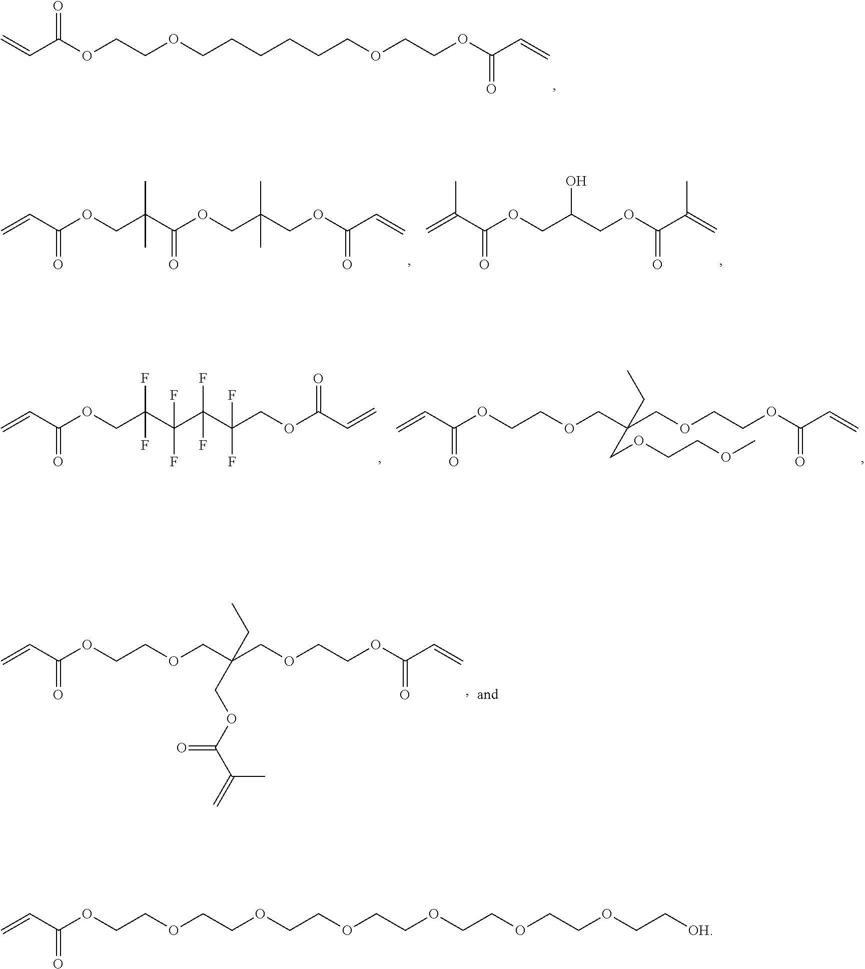

In addition to acrylate monomers, other agents can be used to form a funcfionalized pre-polymer that can be cross-linked by photopolymerization in accordance with various embodiments of the present inventions. Examples of such agents include, but are not limited to, glycidyl, epichlorohydrin, triphenylphosphine, diethyl azodicarboxylate (DEAD), divinyladipate, and divinylsebacate with the use of enzymes as catalysts, phosgene-type reagents, di-acid chlorides, bis-anhydrides, bis-halides, metal surfaces, and combinations thereof.

It is to be understood that, in various embodiments, vinyl groups can be incorporated in the backbone of the pre-polymer using, e.g., free carboxyl groups on the pre-polymer. For example, hydroxyethyl methacrylate can be incorporated through the COOH groups of the pre-polymer using carbonyl diimidazole activation chemistry.

Vinyl groups can be incorporated in the backbone of the pre-polymer with or with-out the use of catalyst, although the use of a catalyst is preferred. A wide variety of catalysts can be used in various embodiments, including, but not limited to, 4-(dimethylamino)pyridine, N-hydroxy succinimide, carbodiimides, and pyridine. Preferably, the reaction is carried out in a solvent, examples of suitable solvents include, but are not limited to, benzene, toluene, chloroform, dichloromethane, ethyl acetate, and tethrahydrofuran.

In various embodiments, acrylation of the pre-polymer can be carried out by reacting the pre-polymer with acryloyl chloride (in the presence of triethylamine and 4-(dimethylamino)pyridine (4-DMAP) as catalysts) in anhydrous dichloromethane. Using these reagents it is preferred that that this reaction is carried out under extremely dry conditions. An example of a resultant acrylation is schematically illustrated in FIG. 3B. It is to be understood that not all binding possibilities and resultant products are shown in FIG. 3B. For example, although it is believed that the backbone OH groups of the pre-polymer are preferentially acrylated, the carboxylic acid groups can also be modified.

The degree of acrylation of the pre-polymer can be used to adjust the properties of the resultant cross-linked polymer. Accordingly, in various aspects the present inventions provide methods for formation of elastomeric polymers with specific physical and mechanical properties. In various embodiments, one or more of the degree of acrylation and the use of substituents on the acrylate groups can be used to control properties such as degradation and swelling and mechanical properties.

The molar ratio of acryloyl chloride to available hydroxyl groups can be varied to adjust the degree of acrylation. In various embodiments, the acrylated pre-polymer is a viscous liquid that can be cured without solvent. Accordingly, in various embodiments, the present inventions provide methods for in vivo curing of the acrylated pre-polymer to form a elastomeric biodegradable composition or material.

### Photopolymerization

In various embodiments, the acrylated pre-polymers into a polymeric network using a free radical initiated reaction, such as, for example, by photoinitiated polymerization, photopolymerization. In various preferred embodiments, acrylated pre-polymer is irradiated with light (typically ultraviolet (UV) light) in the presence of a photoinitiator to facilitate the reaction. Examples of suitable photoinitiators include, but are not limited to: 2-dimethoxy-2-phenyl-acetophenone, 2-hydroxy-1-[4-(hydroxyethoxy)phenyl]-2-methyl-1-propanone (Irgacure 2959), 1-hydroxycyclohexyl-1-phenyl ketone (Irgacure 184), 2-hydroxy-2-methyl-1-phenyl-1-propanone (Darocur 1173), 2-benzyl-2-(dimethylamino)-1-[4-morpholinyl)phenyl]-1-butanone (Irgacure 369), methylbenzoylformate (Darocur MBF), oxy-phenyl-acetic acid-2-[2-oxo-2-phenyl-acetoxy-ethoxy]-ethyl ester (Irgacure 754), 2-methyl-1-[4-(methylthio)phenyl]-2-(4-morpholinyl)-1-propanone (Irgacure 907), diphenyl(2,4,6-trimethylbenzoyl)-phosphine oxide (Darocur TPO), phosphine oxide, phenyl bis(2,4,6-trimethyl benzoyl) (Irgacure 819), and combinations thereof. In various preferred embodiments, acrylated pre-polymer is irradiated with visible light (typically blue light) in the presence of a photoinitiator to facilitate the reaction. Examples of photoinitiators for visible light include camphorquinone among others.

In various embodiments, e.g., in vivo photopolymerization and other medical applications, the use of cytocompatible photoinitiators is preferred and may be require by regulatory agencies. It has been reported that the photoinitiator Irgacure 2959 causes minimal cytotoxicity (cell death) over a broad range of mammalian cell types and species.

### Cross-Links and the Polymer Network

It is to be understood that in the formation of a polymer network that the links and polymer strands of the network are not homogeneous. For example, FIGS. 3C and 3D schematically illustrate examples of portions of the polymer network formed by the photopolymerization methods of the present invention using PGSA.

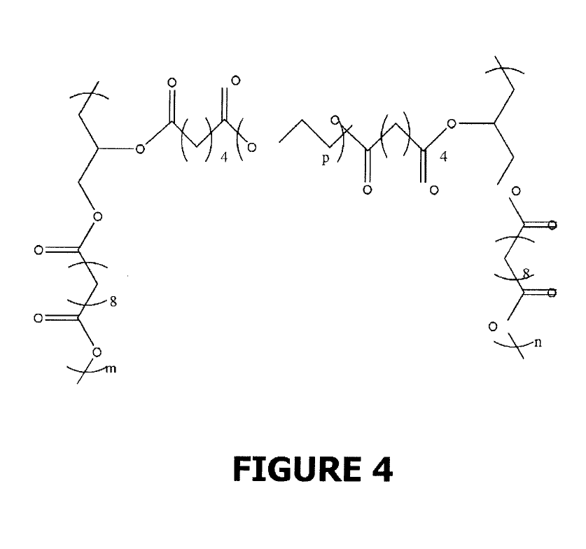

In various aspects of the present invention, the formation of different cross-links in the polymer network is exploited to adjust, or even “tailor” the properties of the resultant polymer. For example, FIG. 4 schematically illustrate examples of portions of the polymer network formed by the photopolymerization methods of the present invention using PGSA and PEGD, it being understood that cross-links substantially as illustrated in FIGS. 3C and 3D are also present in the PGSA-PEG polymer network.

### “Co-Polymer” Networks

In various aspects, the present inventions provide elastic biodegradable polymer compositions and materials formed from an acrylated pre-polymer of the present inventions and one or more additional molecules (referred to as co-polymers herein) functionalized to the acrylate of the acrylated pre-polymer and/or a hydroxyl group of the acrylated pre-polymer. A wide variety of co-polymers can be used including, but not limited to, one or more hydrogel or other polymeric precursors (e.g., precursors that may be modified to contain acrylate groups such as poly(ethylene glycol), dextran, chitosan, hyaluronic acid, alginate, acrylate based precursors including, for example, acrylic acid, butyl acrylate, 2-ethylhexyl acrylate, methyl acrylate, ethyl acrylate, acrylonitrile, n-butanol, methyl methacrylate, and TMPTA, trimethylol propane trimethacrylate, pentaerythritol trimethacrylate, pentaerythritol tetramethacrylate, ethylene glycol dimethacrylate. dipentaeiythritol penta acrylate, Bis-GMA (Bis phenol A glycidal methacrylate) and TEGDMA (tri-ethylene, glycol dimethacrylate), sucrose acrylate, etc. and combinations thereof can be reacted with the acrylated pre-polymer (e.g. PGSA) prior to or during free radical polymerization to modify the cross-links between the polymer chains.

In various aspects, the present inventions provide elastic biodegradable polymer compositions and materials formed by the reaction of a multifunctional alcohol or ether (that is a compound having two or more OR groups, where each R is independently H and an alkyl) and a difunctional or higher order acid (e.g., a diacid) to form a pre-polymer (see, e.g., FIG. 3A). In various embodiments, at least a portion of the pre-polymers are functionalized with a vinyl group to form a mixture of acrylated pre-polymers which are reacted with one or more co-polymers to form. It is to be under stood that the co-polymer can be added before acrylation of the pre-polymer, during the acrylation reaction, after to the acrylated pre-polymer, or a combination thereof. The resultant mixture is then photopolymerized to form the polymer network. In various preferred embodiments, the co-polymer is acrylated and the acrylated co-polymer combined with the acrylated pre-polymer. In various embodiments, the acrylation of the co-polymer and/or prepolymer with an assymetrical monoacrylate molecules (e.g. Acryloyl-poly(ethylene glycol)-N-hydroxy succinimide) provides, for example, an anchoring moiety that can be further modified (e.g., addition of cell-adhesive molecules).

In various aspects of the present invention, the formation of different cross-links in the polymer network is exploited to adjust, or even “tailor” the properties of the resultant polymer. For example, in various embodiments two or more types of cross-links (e.g. numbers of carbons, different types of groups, e.g., aromatic groups being more rigid, etc.) are used to adjust the properties of the resultant polymer network. In various embodiments, an acrylated pre-polymer (e.g., PGSA) can be combined with a co-polymer (e.g. PEG) in proportions to provide, e.g., one or more of swelling control, degradation control and anti-fouling of the crosslinked polyester.

In various embodiments, a liquid acrylated pre-polymer matrix is combined with acrylated hydrogel precursors to impart mechanical, biodegradable, and swelling properties that are not normally associated with typical hydrogel materials. For example, a hydrogel formed from 20% (w/w) poly(ethylene glycol)di-acrylate (PEGD, 700 Da) in water exhibits an elongation of 14%, Young's modulus of 0.54 MPa and ultimate strength of 0.063 MPa. Through combining PEG with PGSA (DA=0.5), the Young's modulus, ultimate strength, elongation and swelling ratio can be precisely controlled. With increasing acrylated pre-polymer concentration the elongation ranged from 4 to 60%, Young's modulus from 20 to 0.6 MPa and ultimate strength from 0.890 to 0.270 MPa. The networks formed by the copolymerization of PEGD with acrylated pre-polymer (DA=0.5) (50:50) showed a ten fold higher Young's modulus and ultimate strength than the typical PEGDA hydrogel while maintaining its elongation at break . Increased elongation was found in materials containing greater then 50% PEGDA. Also, the swelling behavior of these networks can be tuned from 40% to 10% through changing the concentration of acrylated pre-polymer between 10% and 90%. PGSA elastomeric networks are degradable at physiologic conditions and show cell-adhesive and non-cytotoxic properties. As can be seen, the present invention in various embodiments can provide materials and compositions where the degradation rate can be increased without necessarily decreasing the mechanical strength because, it is believed with out being held to theory, of the incorporation of two or more types of cross-links. As it can also be seen, the present invention in various embodiments can provide a degradation rate that is substantially independent of overall crosslink density and/or substantially independent of overall crosslink density within a range of overall crosslink densities.

### Forms and Fabrication of Various Morphologies

The liquid acrylated pre-polymers, and acrylated pre-polymer/co-polymer compositions of the present invention be processed into a wide range of formats and geometries. The acrylated pre-polymer can used to manufacture nanoparticles and/or microparticles of the compositions and materials of the present inventions, which was previously not possible with, e.g., PGS due to the processing conditions (thermal curing). In various embodiments, such particles can be used for the controlled release of drugs, e.g., in joints or other mechanically dynamic environments. The acrylated pre-polymer can used to manufacture very thin walled tubes of the compositions and materials of the present inventions; the tube having an inner diameter of about 1 mm and an about 0.20 mm wall thickness. In various embodiments, such tubes cab be used, e.g., as small-diameter vascular grafts were made. The acrylated pre-polymer can processed to provide compositions and materials of the present inventions having micropatterned surfaces, and porous scaffolds. The acrylated pre-polymer can also be processed into thicker (>6 mm) geometries. For example, 20 mm thick geometries were fabricated, which was previously not possible with thermally cured PGS, due to bubble formation. In various embodiments, the ability to form materials and compositions of the present invention into thicker structures without substantial bubble formation, facilitates the formation of complex structures.

### Methods of Fabrication

In various aspects the present inventions provide methods of forming biodegradable elastomeric compositions, materials and devices. In various embodiments, to fabricate photocurable biodegradable elastomers at room temperature, the following process can be employed. (1) a pre-polymer, e.g., from glycerol and sebacic acid, is created; (2) functional hydroxyl groups on backbone of the pre-polymer are acrylated and the reaction product subsequently purified; and (3) the acrylated pre-polymer was is photopolymerized with UV light in the presence of a photoinitiator. Where glycerol and sebacic acid is used to form the pre-polymer, the resultant elastomer is referred to as poly(glycerol sebacate adipate) PGSA. In various embodiments, a PGS pre-polymer had a weight average molecular weight (Mw) of 23 kDa and a molar composition of approximately 1:1 glycerol:sebacic acid. To functionalize the pre-polymer with vinyl groups, it can be reacted with different molar ratios of acryloyl chloride, at room temperature.

In various embodiments, where glycerol and sebacic acid is used to form the pre-polymer and acrylation is by acryloyl chloride, the degree of acrylation (DA) increases substantially linearly when the molar ratio of acryloyl chloride to glycerol-sebacate can be varied from 0.3 to 0.8 (see, e.g., FIG. 10) and increasing the DA in PGSA from 0.3-0.8, the can increase the crosslink density, for example, from about 6 to about 185 mol/m.sup.3 and the relative molecular mass between crosslink can be decreased.

In various aspects, to fabricate biodegradable elastomers at room temperature, provided are methods using one or more of a Mitsunobu-type reaction, polymerization using a thermal initiator, redox-pair initiated polymerization, Michael-type type addition reaction using a bifunctional sulfhydryl compound, to cross-link the pre-polymers.

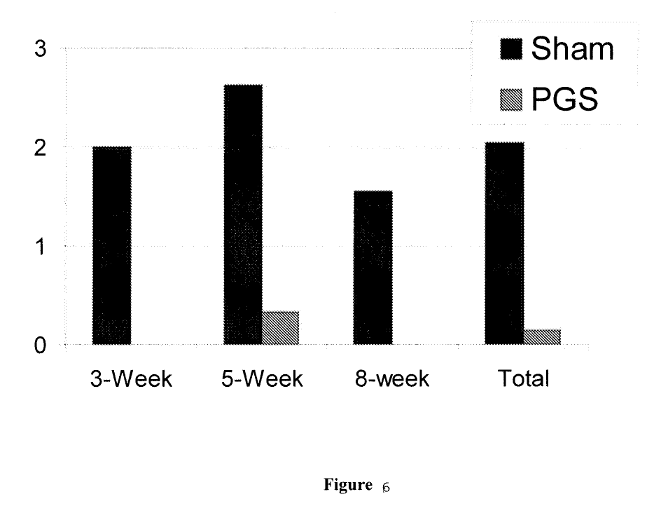

In various embodiments, a Mitsunobu type reaction is used to cross-link the pre-polymer. For example, referring to FIG. 6A, a PGS pre-polymer dissolved in THF is reacted, at room temperature and pressure conditions, with diisopropyl azodicarboxylate and triphenylphosphine. Within about 1 hour of reaction time the final elastomeric cross-linked polyester composition product was formed. The mild conditions of this reaction, for example, also permit the incorporation of a variety of functional groups, such as, e.g., esters, epoxides, halides into the elastomeric cross-linked polyester composition.

In various embodiments, mono-acids can be used to introduce ester linked side-chains, and mono-alcohols can be used to create ether linked side-chains. In various embodiments, poly-beta amino esters, can be created, a class of biomaterials that have shown promise in gene delivery. One potential limitation in the development of poly-beta amino esters for clinical applications is the inability to synthesize high molecular weight products. The application of the Misunobu-type reaction of the present inventions could be useful in overcoming this obstacle to produce high molecular weight formulations by crosslinking side chains. In various embodiments, the present inventions thus include, particles for gene delivery comprising poly-beta amino ester microspheres.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

### Materials and Methods

The PGS pre-polymer was synthesized by polycondensation of an equimolar mixture of anhydrous glycerol and sebacic acid as previously reported (Sundback C. A., et al., Biomaterials, 26:27, 5454-5464 (2005). The PGS prepolymer was melted quickly at 120° C., poured into an open mold based on weight, and cross-linked at 120° C. and 40 mTorr vacuum for 66 hours. The resulting films were 0.7 mm thick. The films were cut into 3 cm×3 cm squares. Unreacted mono/oligomers were removed using decreasing concentrations of ethanol, washed liberally with MillQ water, soaked in water overnight, and dried for 24 hr at 60° C. under vacuum. The films were sterilized by autoclave, prior to use.

The efficacy of PGS film to prevent adhesions was tested in a pilot study using the rat model cecal abrasion and abdominal wall defect technique (Harris E. S., et al., Surgery, 117:6, 663-669 (1995). Six Lewis rats underwent a midline laparotomy and a 1 cm×2 cm parietal peritoneal defect was created bluntly. The cecum was then mobilized and positioned so that it would contact the defect at closure. The cecum was abraded until petechial bleeding was observed. Each defect was exposed to air for 10 minutes. One PGS film was then implanted into the peritoneal cavity, between the visceral organs and the anterior abdominal wall, of 3 animals forming the study group. Control animals did not receive a PGS implantation. The animals were weighed at regular intervals and observed for signs of small bowel obstruction including obvious distress, abdominal distension, and obstipation. All animals were sacrificed at 28 days and their abdomens were inspected for the presence of VP adhesions.

### Example 2

### Results and Discussion

The formation of peritoneal adhesions is caused by the apposition of two areas of damaged or inflamed peritoneum. Specifically, a VP adhesion occurs when an area of damaged or inflamed visceral peritoneum adheres to an area of damaged or inflamed parietal peritoneum. The goal of the study was to demonstrate that a PGS film could be used to prevent such an apposition and subsequently the development of VP adhesions. No animal in the study group developed a VP adhesion. The study animals gained an average of 111 g over the study period and never demonstrated any signs or symptoms of gastrointestinal distress. Upon gross examination, each implant was found in the left paracolic gutter at a position of dependency, indicating a high degree of mobility throughout the study period. Each implant was covered with a thin fibrous capsule and one implant was surrounded by omentum with a re-absorbing hematoma.

Two control animals developed lethal VP adhesions and the remaining animal showed the development of dense adhesions at sacrifice. The presence of dense cecal-cecal adhesions in all Animals served as an internal control to demonstrate the presence of a normal adhesion forming mechanism.

In a rat model, a PGS film prevented the formation of a VP adhesion over a period of 28 days. This initial demonstration of efficacy indicates that PGS films warrant further evaluation as a preventative barrier to the formation of VP adhesions. Development of such a barrier would be especially beneficial for patients who are prone to adhesions and re-operation, such as those with Crohn's disease or acute diverticulitis requiring diversion and an eventual reversal of a colostomy.

### Example 3

### Animal 2; Study 1 Week

The animal was administered 0.2 ml of Donnitor/0.1 ml of Ketamine via IM injection. The animal's hair was removed using a depilatory agent and prepped in the standard surgical fashion and a 0.5 cm midline incision was made in the skin of the abdominal wall. The dissection was carried down to the level of the peritoneum. The peritoneum was incised sharply and the abdominal wall was opened to the extent of the skin incision. The abdominal contents were briefly visually inspected and no adhesions were noted. PGS sample C was implanted as diagrammed in the surgical record. The abdominal wall was re-approximated using a running 4-0 Ethilon suture. The skin was closed using interrupted horizontal mattress sutures of 4-0 silk. The animal was administered 0.1 ml of antesedan for reversal of anesthesia. The animal was placed under a heat lamp during recovery. The animal recovered without incident. This animal seemed notably less uncomfortable post-operatively compared to animal 1 without a PGS barrier film.

### Example 4

### In Vivo Adhesion Prevention

Animals: This study was performed with the Approval of the Subcommitte on Research Animal Care at Massachusetts General Hospital. Forty-two Wistar female rats initially weighing 278 to 372 g were used in this study. The rats were housed in an environment of alternating 12-hour light and dark cycles with foiod and water available ad libitum. A pig, euthanized for a separate study, was used to demonstrate the ease of laparoscopic placement of this device.

Preparation of PGS Films: The PGS pre-polymer was synthesized by polycondensation of an equimolar mixture of anhydrous glycerol and sebacic acid as previously reported³⁴. To prepare 500 μm thick PGS polymer films, silicone wafers were spin-coated with sucrose as a release agent. 500 μm thick silicone rubber masks were affixed to sucrose-coated silicone wafers forming a trough mold. A 10 w/v % PGS prepolymer solution in tetrahydrofuran was pipetted into the trough spread until level with the surrounding silicone rubber forming PGS prepolymer films were 500 μm thick. The films were dried overnight and then cross-linked at 120° C. and 40 mTorr vacuum for 66 hours. Unreacted mono/oligomers were removed using decreasing concentrations of ethanol. The films were cut into 3 cm×3 cm squares; washed liberally with PBS; soaked in PBS overnight and sterilized by autoclave.

In Vivo Adhesion Prevention Study: The efficacy of PGS film to prevent adhesions was tested using the rat model cecal abrasion and abdominal wall defect technique. Forty-two rats were picpped and draped in the standard surgical manner and underwent a midline laparotomy. A 1 cm×2 cm parietal peritoneal defect was created overlying the anatomic position of the cecum using sharp abrasion. The cecum was then mobilized and abraded until petechial bleeding was observed. Each defect was exposed to air for 10 minutes. The abdominal wall was closed using 4-0 Etihlon (Ethicon, Somerville, N.J.) (control) after one PGS film was implanted into the peritoneal cavity, between the visceral organs and the anterior abdominal wall. The skin was closed with a horizontal mattress technique using 4-0 silk sutures. The animals were weighed at regular intervals and observed for signs of small bowel obstruction including obvious distress, abdominal distension, and obstipation. Animals were sacrificed at 3, 5 and 8 weeks and their abdomens were inspected for the presence of VP adhesions. Altogether 3 Groups were evaluated in this study: 6 animals at 3 weeks, 18 animals at 5 weeks and 18 animals at 8 weeks.

Evaluation of V-P Adhesions: At the indicated timepoints, animals were euthanized via lethal overdose of xylazine and Phenobarbital. A transverse incision was made just caudal to the xiphoid process. A skin flap was developed and the skin was removed from the entire abdominal wall. The abdominal wall dissection was carried into the peritoneal cavity in a transverse fashion. The peritoneal cavity was inspected for the presence of gross adhesions and every abdomen was photographed prior to further dissection. The abdominal wall was then raised as a flap via bilateral dissection along the mid-axillary line from the costo-chondral margin to the pelvic brim. The presence of V-P adhesions was recorded and each adhesion was scored (See FIG. 2) using the following scale:

| Score | Description |
|---|---|
| 0 | No Adhesions |
| 1 | Filmy, Thin, Avascular |
| 2 | Limited Vascularity, Moderate Thickness |
| 3 | Well Vascularized, Dense Thickness |

Evaluation aliglammara All PGS remnants were identified and collected. Samples for histologic examination were obtained from the spleen, liver, colon, small bowel, abdominal wall, and adhesions when present. All specimens were washed in PBS and fixed in formalin overnight. All PGS remnants were separated from any overlying capsule and stored in water for degradation evaluation. PGS capsules and all tissue specimens were paraffin-embedded and stained with Hematoxylin & Eosin.

Demonstrations of Laparoscpic Placement: Immediately following euthanasia by injection for a separate study, a 1.2 cm supra-umbilical incision was made in the midline and the dissection was carried down to the level of the peritoneum. The peritoneum was entered under direct visualization and an Autosuture Bluntport 12 mm trocar (Covidien Norwalk, Conn.) was placed in the supra-umbilical position. The trocar was fixed using 1 Prolene sutures and the abdomen was insufflated to a pressure of 15 mm Hg. A 10 mm 30⁰ laparoscope was placed through the trocar and the abdominal contents were observed to be normal. The left lower quadrant of the abdominal wall was trans-illuminated and a small skin incision was made lateral to the inferior epigastric vessels. A 12 mm Autosuture Versaport trocar was passed through the incision and into the peritoneum under laparoscopic visualization. The camera replaced in this trocar. The inner trocar of the supra-umbilical Bluntport was removed from the threaded anchoring device and taken to the back table. An atraumatic grasper was passed through the seal of the Bluntport trocar. A wetted sheet of 500 μm thick PGS measuring 6 cm×6 cm was rolled and grasped with the grasper and retracted into the cannula of the trocar. The trocar was replaced into the abdomen through its anchoring device and the grasper was advanced into the abdomen. A second 500 μm thick sheet of PGS was passed into the peritoneal cavity in a similar fashion. All laparoscopic maneuvers were digitally recorded in MPEG2 video format using a digital recording system to demonstrate the ease in handling this material.

Statistical Analysis: The incidence of Animals with adhesions and the scores of the adhesions were compared between the study and control groups using the Fischer exact test. A P value of <0.05 was considered significant.

Results: In vivo animal studies were carried out to evaluate the performance of a PGS film to prevent the formation of post-operative adhesions. Using the method described above, we created films of PGS with an average thickness of 600 μm ranging from 510 μm to 620 μm. At 3 weeks, 66% of the sham animals had V-P adhesions (2/3), compared to no V-P adhesions were found in the study group (0/3). The animals evaluated at 5 week had an adhesion rate of 88% in the control group (7/8, 1 death) compared with 11% in the study group (1/9). The 8 week animals had an adhesion rate of 67% in the sham animals (6/9) and no V-p adhesions were observed in the study group (0/9). The total study adhesion rate was 75% in the control animals and 5% in the study animals (p<0.0000001). This represents a reduction in adhesion formation of 94% in the animals that received PGS film implantations. Adhesion severity was graded for each group and the control animals had an average severity score of 2.0, 2.6, 1.6 and 2.1 in the 3, 5, 8 and Total groups respectively. Only one adhesion was observed in all the animals of the study groups yielding an average severity score of 0, 0.33, 0, and 0.14 in the 3, 5, 8 and Total groups respectively.

The remnants of each PGS film in the 3- and 5-week studies were identified, however only 2 animals had any PGS remnant in the 8-week study. Each remnant was covered with a thin fibrous capsule and each remnant typically formed a broad attachment to the otherwise mobile omentum. The 8-week study animals with no PGS remnant all had a small structure presumed to be a PGS capsule remnant attached to the omentum which was removed and histologically examined. All capsule sections showed minimal tissue response, composed of mesothelial cells with the absence of inflammatory or giant cells. In addition, the parietal peritonea were evaluated and found to be normal in all cases.

All V-P adhesions in the control animals were removed en bloc with the abdominal wall and inspected histologically. All adhesions were dense, well-vascularized structures containing collagen and bridging the sub-mesothelial space between the abdominal wall and the cecum. Samples were also obtained from the spleen, liver, colon, and small bowel. No samples had evidence of inflammation or necrosis and were found to be normal.

Using standard laparscopic surgical equipment and techniques we demonstrated that a PGS film can easily be placed in the abdominal cavity. Two sheets were placed, and then re-positioned multiple times in the right lower quadrant. In addition, several rents were made in the parietal peritoneum. A sheet of PGS was than positioned to cover the rents and repositioned multiple times. These maneuvers demonstrated that PGS films can be positioned and re-positioned on moist tissue surfaces without injury to the tissue or loss of barrier material using basic laparoscopic techniques.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of preventing adhesions between two tissue surfaces comprising:
providing a film comprising a condensation polymer of glycerol and a diacid;
positioning said film between a first tissue surface and a second tissue surface under conditions effective to prevent adhesion between said first tissue surface and said second tissue surface.

2. The method of claim 1, wherein the diacid is sebacic acid.

3. The method of claim 1, wherein the polymer is polyglycerol sebacate.

4. The method of claim 1, wherein the first tissue surface and/or second tissue surface is a site of surgical activity.

5. The method of claim 1, wherein the first tissue and/or second tissue is visceral tissue.

6. The method of claim 1, wherein the first tissue and/or second tissue is peritoneal tissue.

7. The method of claim 1, wherein the adhesions prevented are viscero-parietal peritoneal adhesions.

8. A method of preventing adhesions between two tissue surfaces comprising:
providing a film consisting essentially of a condensation polymer of glycerol and a diacid; and
positioning said film between a first tissue surface and a second tissue surface under conditions effective to prevent adhesion between said first tissue surface and said second tissue surface.

9. The method of claim 8, wherein the diacid is sebacic acid.

10. The method of claim 8, wherein the polymer is polyglycerol sebacate.

11. The method of claim 8, wherein the first tissue surface and/or second tissue surface is a site of surgical activity.

12. The method of claim 8, wherein the first tissue and/or second tissue is visceral tissue.

13. The method of claim 8, wherein the first tissue and/or second tissue is peritoneal tissue.

14. The method of claim 8, wherein the adhesions prevented are viscero-parietal peritoneal adhesions.

15. The method of claim 8, wherein the film comprises bioactive agents, drugs, or other molecules.

16. The method of claim 8, wherein the film comprises surface-deposits of a material to enhance bonding of the film to a tissue.

17. The method of claim 8, wherein the film does not contain anti-inflammatory drugs.

18. The method of claim 1, wherein the film comprises bioactive agents, drugs, or other molecules.

19. The method of claim 1, wherein the film comprises surface-deposits of a material to enhance bonding of the film to a tissue.

20. The method of claim 1, wherein the film does not contain anti-inflammatory drugs.
